(19) United States
(12) Patent Application Publication
(10) Pub. No.: US 2010/0087819 A1
(43) Pub. Date: Apr. 8, 2010

(54) Forward Kinematic Solution for a Hexapod Manipulator and Method of Use

(75) Inventor: Michael W. Mullaney, Kinnelon, NJ (US)

Correspondence Address: HAYNES AND BOONE, LLP;IP Section, 2323 Victory Avenue, Suite 700, Dallas, TX, 75219, US

(73) Assignee: EXTRAORTHO, INC., Memphis, TN (US)

(21) Appl. No.: 12/575,191

(22) Filed: Oct. 7, 2009

Related U.S. Application Data
(60) Provisional application No. 61/103,524, filed on Oct. 7, 2008.

(51) Int. Cl. A61B 17/62; A61B 17/64

(52) U.S. Cl. 606/56; 606/54

(57) ABSTRACT

The present disclosure provides methods, systems, and computer program product for treating a fractured bone using a hexapod manipulator. The hexapod manipulator has a first ring and a second ring, with the first and second rings being connected by six telescopic struts. The method includes the steps of determining a current position of the second ring relative to the first ring; determining a desired position of the second ring relative to the first ring; and computing with a processing system the difference between the current position and the desired position of the second ring relative to the first ring using a linear approach to a nonlinear problem.

### CROSS-REFERENCE TO RELATED APPLICATION

This application is a non-provisional of U.S. Patent Application No. 61/103,524, titled “Forward Kinematic Solution for a Hexapod Manipulator and Method of Use,” filed on Oct. 7, 2008, incorporated herein by reference.

### FIELD OF THE INVENTION

This disclosure is directed to a method of aligning bone segments with a hexapod manipulator, and more particularly, a method of aligning bone segments by solving a forward kinematic solution that allows for the domination of manipulator strut adjustments.

### BACKGROUND

In orthopedic medicine, physicians often need to correct certain skeletal injuries or deformities with external fixator devices. These devices use pins or wires attached to the separate bone segments and an external structural frame to align, or fix, the bone segments in a way to aid in repairing the injury or correcting the deformity. Often the physician must gradually adjust the orientation of the bone segments over time, optimally with the capability to adjust the orientation along six degrees of freedom to ensure the bone segments are placed in the correct anatomic condition.

Devices and methods for treating musculoskeletal deformities are well known in the art. One broad category of devices is a circular ring device category exemplified by Ilizarov-type systems, which have two rings connected by linear struts with a fixed or hinged connection at each end of each strut. A device called a space frame or hexapod manipulator has two rings connected by six linear struts having a spherical joint at each end arranged in a hexapod configuration.

These hexapod manipulators are parallel devices with prismatic joints that control the degrees of freedom of a moving frame with respect to a stationary frame. Because these joints all connect to both frames rather than connecting to each other in some serial fashion, any given adjustment to any of the six struts will result in a change to all six degrees of freedom. This characteristic makes the hexapod manipulator less intuitive to use. Further, this makes the calculation of effects of changes in the Inverse Kinematic Solution (IKS) rather trivial while making calculation of effects of changes in the Forward Kinematic Solution (FKS) exceedingly difficult without the use of numerical methods. Therefore, a computer program is often required to direct the user in making the adjustments to correct the deformity.

One conventional way of calculating the effects of changes in the FKS is inputting data representing a current or initial position of a hexapod manipulator into a CAD system having the three dimensional data of the hexapod, inputting a desired position of the hexapod to align a patient's bone, and using the CAD system to output instructions for manipulating the hexapod from the initial position to the desired position. However, CAD systems for such calculations are expensive and providing such a system in operating rooms in many hospitals is not practicable. Further, remote CAD systems accessible over the Internet from an operating room have their own risks, including that all licenses would be tied up by other surgeons across the country, that access may be limited by Internet unpredictability, maintenance on the remote system, confidentiality, and others.

What is needed is a method of simplifying adjustment of hexapod manipulators to treat bone segments using non-specialized processing systems.

### SUMMARY

In order to address one or more shortcomings in the prior art, this disclosure is directed to methods, systems, and computer program product for simplifying surgical implantation and adjustment of hexapod manipulators. Some embodiments simplify implantation by simple, locally performed calculation of the FKS of a hexapod manipulator using a standard processing system, such as a desktop, laptop, PDA, or another non-specialized processing system already found in doctor's offices, operating rooms, recovery centers, outpatient facilities and other healthcare facilities.

The forthcoming methods, systems, and computer program product represent an analytical linear approach to a non-linear problem involving hexapod frames. This utilizes a Lagrangian approach whereby the nodal positions are described with respect to a fixed Cartesian coordinate system in space. The nodal strains are determined based on an incremental approach to the loading. At each step a small fraction of the anticipated deflection of interest is applied, strains are calculated based on the principle of minimum total potential energy resulting in a balance of forces and a balance of energy between that which is applied in terms of applied force, based on an after determined stiffness of the system, through a distance, the potential energy of the applied forces, and the summation of the energy stored with the individual elements as the total strain energy.

In order to integrate the energy stored within the structure, the individual elements of the structure are mapped into bar elements having end nodes with positions known with respect to the global coordinate system. Once this is achieved, the longitudinal strains can then be computed based on the nodal strains being mapped into a local element coordinate system. To obtain the corresponding equilibrium statement, the total potential energy of the nodal parameters may be differentiated with respect to each of the Cartesian directions of the local element coordinate. To accomplish this, it is intended to develop the derivatives with the integrals and integrate them analytically. The 6 nodal positions with three degrees of freedom per node can be represented in vector notation whereby:

1. One can assume an initial non-deformed position,

2. Determine the stored energy based on the applied nodal deflections,

3. Calculate the gradient at the initial position,

4. Determine the element deflection, and

5. Update the position until the deflection is sufficiently small, i.e. gradient approaches zero.

The incremental nature of the approach lends itself to the treatment of non-linearities in so far as at each step of the process the engineering stress can be calculated. Similarly the stiffness of the element can be updated on a step wise basis leading to a quasi-linear treatment of a non-linear problem.

The approach is effective as it is able to address non-linear effects in the same way by allowing the manipulation of the element stiffness on a step wise basis whereby existing elements along with the minimization of potential energy approach can be used in a step wise fashion to model real applications. Essentially this can be likened to an iterative solution where the model changes based on the results of the previous iteration.

In a first exemplary aspect, the present disclosure is directed to a method of treating a fractured bone using a hexapod manipulator. The hexapod manipulator has a first ring and a second ring, with the first and second rings being connected by six telescopic struts. The method includes the steps of determining a current position of the second ring relative to the first ring; determining a desired position of the second ring relative to the first ring; and computing with a processing system the difference between the current position and the desired position of the second ring relative to the first ring using a linear approach to a nonlinear problem.

In some aspects, the method includes constructing with the processing system a neutral frame having a Moving Reference ring and a Fixed Reference ring with nodal locations representing ball joints on the hexapod manipulator, and calculating the nodal locations of the Moving Reference ring using a global stiffness matrix K. Some aspects include outputting at least one of: the difference in strut lengths between the current position and the desired position, and a strut set value representative of the desired position. Other aspects include determining the forces and displacements of a single element in both an element coordinate system and in a global coordinate system, the element coordinate system relating to a single strut position and the global coordinate system being the coordinate system used to reference the manipulator. In some aspects, computing with a processing system the difference between the current position and the desired position includes the steps of setting applied forces equal to zero and introducing a small strain into the struts and solving for nodal deflections. In some aspects this includes updating the nodal positions to reflect new positions as a result of the deflections and again solving for nodal deflections at the new positions.

In another exemplary aspect, the present disclosure is directed to a method of determining the difference between a first initial position of a hexapod fixator and a second desired position of the fixator. The fixator has six struts extending between and connecting two fixator rings. The method includes the steps of generating a first digital x-ray image of a plurality of bone segments and the hexapod fixator and generating a second digital x-ray image of a plurality of bone segments and the hexapod fixator, the second digital x-ray image being taken from an angle relative to the first digital x-ray image. The angle can be any acute angle, recognizing that as the angle increases, the accuracy also increases because at small angles, the ability to resolve three Cartesian dimensions from the pair of 2D x-ray images decreases. In one example, the angle is ninety degrees, providing a side lateral image relative to the first x-ray image. The method also includes receiving data indicative of the lengths of the six struts of the hexapod manipulator. A first position of nodes representative of the connection location of the rings and struts of the hexapod manipulator is determined relative to a coordinate system in space and the struts are deflected a first amount in the direction of the desired position of the manipulator. The method includes determining a second position of nodes representative of the connection location of the rings and deflected struts of the hexapod manipulator relative to the coordinate system in space and deflecting the struts a second amount in the direction of the desired position of the manipulator.

In some aspects, the method includes repeating the determining and deflecting steps until the determined position of nodes representative of the connection location of the rings and struts corresponds to the second desired position of the fixator, the determined position of the nodes being the final position of the nodes. Some aspects include calculating the difference between the first position of the nodes and the final position of the nodes and outputting the difference to a health care provider. In some aspects, deflecting the struts is accomplished independent of loading applied to the struts. In some aspects, the step of determining the first position of nodes includes: determining a first rotation of a bar element representative of a single strut in both an element coordinate system relating to only the bar element and a global two-dimensional coordinate system relating to the hexapod manipulator; and determining a second rotation of the bar element about a single axis in the global coordinate system, the single axis being different than the two dimensions used to determine the first rotation of the bar element.

In some aspects, the method includes the step of computing a global stiffness matrix for the manipulator and setting applied forces to zero to introduce small strain for the condensed system. In some aspect, the method includes the step of determining the first and second rotations for each of the six struts. In some aspects, the method includes the steps of: introducing strain deflections; and calculating a global stiffness matrix to determine the level of nodal deflections.

In another exemplary aspect, this disclosure is directed to an apparatus comprising a tangible computer-readable storage medium storing a computer program for execution by at least one processor, wherein the program determines a position of a hexapod manipulator having six struts. The program when executed, receives input data representative of initial strut lengths; determines an initial orientation a first ring relative to a second ring based in part of the received input data; establishes a Modulus and cross sectional area for the struts; computes a stiffness matrix based in part on the Modulus and cross sectional area of the struts; calculates desired strut lengths that displace the second ring to a desired position; and outputs the calculated desired strut lengths.

In some aspects, the program determines a coordinate transformation of a Moving Reference ring with respect to a Fixed Reference ring of the hexapod manipulator. Some aspects of the program determine nodal positions of the moving reference ring in a global coordinate system and establishing a global stiffness matrix based on a transformation between the global coordinate system and a element coordinate system, the element being representative of a strut of the hexapod manipulator.

In another exemplary aspect, the present disclosure is directed to a method of treating a fractured bone using a hexapod manipulator. The hexapod manipulator has a first ring and a second ring, the first and second rings being connected by six telescopic struts. The method includes placing the first ring about a first bone segment and fixing the position of the first bone segment relative to the first ring and placing the second ring about the second bone segment and fixing the position of the second bone segment relative to the second ring, the first and second rings being connected at ball joints by the struts. A current position of the second ring is determined relative to the first ring and a desired position of the second ring is determined relative to the first ring. Data is input indicative of the strut lengths into a processing system and desired strut lengths are computed with the processing system, the desired strut lengths corresponding with the desired position of the second ring relative to the first ring. Computing desired strut lengths comprises: constructing with the processing system a neutral frame having a Moving Reference ring and a Fixed Reference ring with nodal locations representing the ball joints of the placed hexapod manipulator; calculating the nodal locations of the Moving Reference ring; calculating the individual strut lengths; calculating a rotation angle of the Moving Ring relative to the Fixed Reference ring; establishing Ee Ae products to differentiate stiff and flexible members; computing the global stiffness matrix K for the initial condition; setting the forces equal to zero and introduce a small strain into the struts and solving for nodal deflections; updating the nodal positions to reflect new positions as a result of the deflections. The method also includes adjusting the struts so the actual strut lengths correspond to the desired strut lengths to position the first and second bone segments for healing.

### BRIEF DESCRIPTION OF THE DRAWINGS

Aspects of the present disclosure are best understood from the following detailed description when read with the accompanying figures.

FIG. 1 is an illustration of a perspective view of a hexapod about a long bone.

FIG. 2 is an illustration of an exemplary strut of the hexapod manipulator.

FIG. 3 is a flow chart of a method of treating a patient in accordance with the present disclosure.

FIG. 4 is a schematic of an exemplary spatial frame manipulator.

FIG. 5 is a flow chart of a method of considering the relationship between a) the element forces and displacements in element coordinates and b) the Forces and displacements in the global coordinate system.

FIG. 6 is an exemplary strut with forces and displacements in the global and element coordinates.

FIG. 7 is a flow chart of a method of using a global stiffness matrix to ultimately arrive at a desired bone manipulator orientation.

FIG. 8 is a block diagram of an exemplary processing system.

### DETAILED DESCRIPTION

It is to be understood that the following disclosure provides many different embodiments, or examples, for implementing different features of various embodiments. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

The following are incorporated by reference in their entirety: U.S. Pat. No. 6,030,386 to Taylor et al., French Patent Publication No. 2 576 774 to Pilippe Moniot, filed Feb. 7, 1985, and U.S. Patent Publication No. 2004/0097922 to Mullaney.

This disclosure is directed to methods, systems, and computer program product for simplifying the process of aligning bone segments with a hexapod manipulator. After implanting the manipulator on bone segments, the manipulator struts must be adjusted to properly align the segments. Rather than calculating desired strut lengths of the hexapod manipulator using complex simultaneous calculations of six quadratic equations, the present disclosure provides methods, systems, and computer program product that calculate the desired strut lengths based on a linear or quasilinear set of equations. Accordingly, the calculations are much simpler to perform and do not require specialized equipment, such as a CAD package or its equivalent. Instead, the calculations can be performed on more commonly available and potentially less expensive non-specialized processing systems, such as those already found in hospitals and operating rooms today.

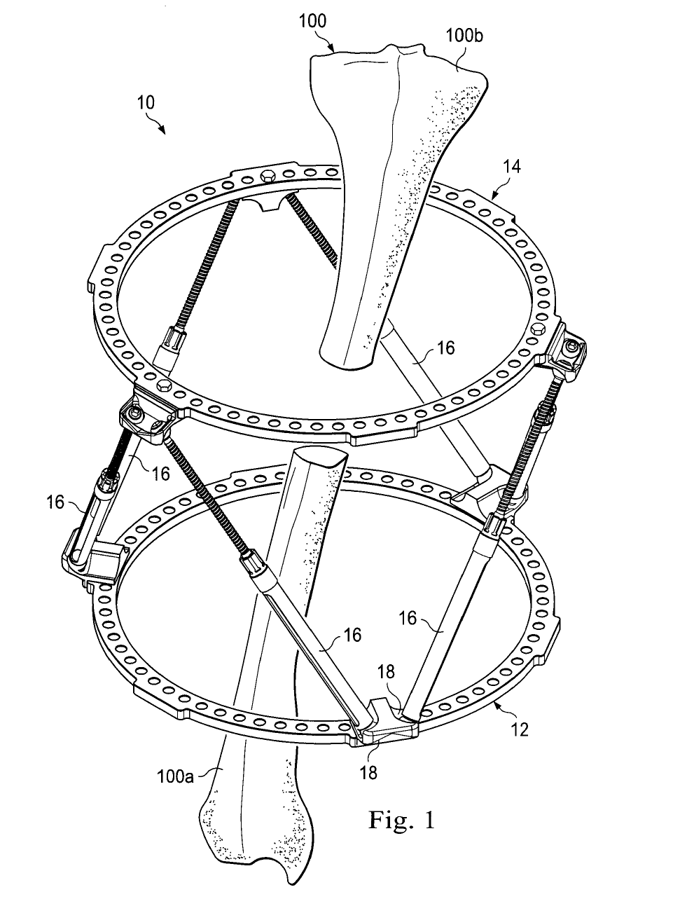

A hexapod manipulator is a space frame with six telescopic struts arranged as a hexapod acting in parallel and yielding an ability to move in all six degrees of freedom. FIG. 1 shows one example of a hexapod manipulator, referenced herein by the numeral 10, disposed about, and fixed with relation to a long bone 100. The hexapod manipulator 10 includes a first ring 12, a second ring 14, and a series of struts 16. The first and second rings 12, 14 form a frame. These are sized to fit around a patient's limb such that during use, the frame is external to the patient's body.

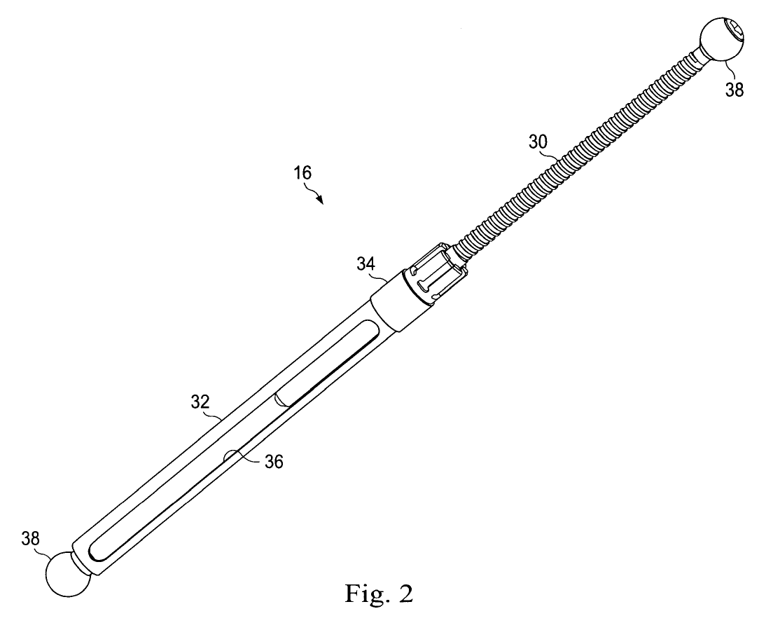

The telescopic struts 16 extend between and connect to both the first and second rings 12, 14 at joints 18. One example of a strut 16 is shown in FIG. 2. The strut 4 has an externally threaded rod 30 which freely extends into a hollow shaft 32. An adjustment member in the form of a nut 34 is rotatably joined to the open end of the hollow shaft 32 and includes internal threads which mate with the threads on the rod 30. The adjustment nut 34 is free to rotate about the axes of the rod 30 and the hollow shaft 32, thereby providing a means for telescopically adjusting the axial positions of the rod relative to the shaft.

An indicator (not shown) of the relative position of the rod 30 to the shaft 32, and thus the effective length of the strut, is provided on the strut 16. In one example, this is a projection in the form of a pin which is radially connected to the rod 30 near the end that extends into the shaft 32. A slot 36 is provided along the side of the shaft 32 so that the indicator may be viewed from outside the shaft 32. The strut 16 may include spherical or spheroid shaped ends or balls 38 that interface with the rings 12, 14 at the joints 18.

Graduation marks (not shown) may be provided alongside the slot 36 to indicate the position of the rod 30 relative to the shaft 32. These may be calibrated, for example, in one millimeter increments, and may indicate the distance between the points of coincident rotation at either end of the strut 16. In some examples, graduation marks indicate the lengths of the strut 16 as an absolute value, rather than the distance from some predetermined neutral position. However, the graduation marks may indicate the percentage of total rod extension, or daily increments for cases where the translation takes place over an extended period of time.

In the exemplary hexapod manipulator shown, the joints 18 are formed in part by the spherically-shaped or spheroid-shaped balls 38. These form nodes that permit the strut 16 to rotate at the joint 18 relative to the ring 12, 14 in any direction. Since the joints 18 are ball joints at the nodes, they are subject only to longitudinal loading. Each strut 16 telescopes to increase or decrease its longitudinal length. The distance between the nodes corresponds to the strut lengths. In use, a surgeon may increase or decrease the strut length by threading one end of the strut relative to the other, sliding the strut ends, or using other known methods for changing the strut length.

The rings 12, 14 connect to the long bone 100 using pins (not shown). These extend radially inward from the rings 12, 14 to penetrate the long bone 100 of the patient's limb and fix the orientation of the bone relative to the rings. As can be seen, the long bone 100 includes two bone segments 100a, 100b. Each bone segment is fixed relative to a single ring by the pins. Changing the length of the struts 16 changes the relative position of the rings 12, 14, which in turn, changes the position of one bone segment relative to the other.

Unlike a serial manipulator such as a robotic arm, a parallel manipulator device like the hexapod manipulator is not easy to assemble mathematically. Interest in utilizing a new, linear or quasilinear approach stems from the realization that the solution to positional problems regarding hexapod manipulators is difficult, at best, to solve directly using non-linear quadratic compatibility equations, such as the system of quadratic distance formulas shown below:

(StrutLength₀)²=(aₓ−BaseRingCoordinates₀)²+(a_{y}−BaseRingCoordinates₁)²+(a_{z}−BaseRingCoordinates₂)²

(StrutLength₁)²=(aₓ−BaseRingCoordinates₃)²+(a_{y}−BaseRingCoordinates₄)²+(a_{z}−BaseRingCoordinates₅)²

(StrutLength₂)²=(bₓ−BaseRingCoordinates₃)²+(b_{y}−BaseRingCoordinates₄)²+(b_{z}−BaseRingCoordinates₅)²

(StrutLength₃)²=(bₓ−BaseRingCoordinates₆)²+(b_{y}−BaseRingCoordinates₂)²+(b_{z}−BaseRingCoordinates₈)²

(StrutLength₄)²=(cₓ−BaseRingCoordinates₆)²+(c_{y}−BaseRingCoordinates₇)²+(c_{z}−BaseRingCoordinates₈)²

(StrutLength₅)²=(cₓ−BaseRingCoordinates₀)²+(c_{y}−BaseRingCoordinates₁)²+(c_{z}−BaseRingCoordinates₂)²

(RingCordLength₁)²=(aₓ−bₓ)²+(a_{y}−b_{y})²+(a_{z}−b_{z})²

(RingCordLength₁)²=(aₓ−cₓ)²+(a_{y}−c_{y})²+(a_{z}−c_{z})²

(RingCordLength₁)²=(cₓ−bₓ)²+(c_{y}−b_{y})²+(c_{z}−b_{z})²

In contrast to calculating using the above complex quadratic equations, the FKS calculation approach disclosed herein assembles the manipulator in computer space to a known configuration and applies incremental displacements to the nodes to solve for the overall loading and in so doing arrives at the overall stiffness. Using stiffness enables a displacement to be determined without the use of a complex quadratic equations shown above.

The system describe herein uses an approach that applies element “strut” strains starting from a known configuration in an incremental fashion until a desired “deformed” state is achieved. Ultimately, the desired deformed state is one where the bone segments are aligned as desired. Along the way, it will also be beneficial to determine the structures stiffness as it applies to a known external load applied over one increment. This calculation approach provides advantages not available from calculation of the system of quadratic equations shown above. The FKS simultaneous solution disclosed herein yields a set of nodal positions for any given set of strut lengths and provides at least the following advantages:
Arrives at a single solution without requiring the addition of Boolean boundary conditions to identify the correct solution among multiple solutions—the solution of the above equations yields multiple solutions without the addition of several Boolean boundary conditions;
Because it determines the solution using a linear system of equation, it is far less complicated and far more efficient from a computational standpoint than the solution to a quadratic system of equations; and
The approach also has the added ability to readily calculate the structural stiffness along the path from initial to the desired “deformed” state.

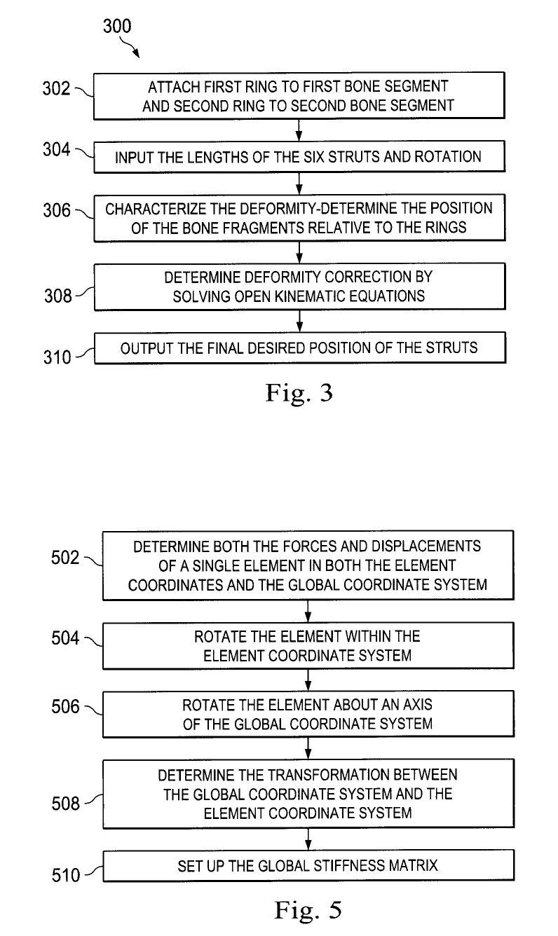

FIG. 3 is a flow chart representing an exemplary method 300 of treating a patient using a hexapod manipulator in accordance with the present disclosure. It should be noted that the method 300 includes steps performed by a surgeon or health care professional, as well as steps performed by a processing system such as a computer. Accordingly, those steps performed by the processing system could be written as one or more sets of instructions stored on a computer-readable medium that could be executed by the processing system. This processing system may include a memory storage device; one or more computer processing units; input devices, such as a keyboard and pointing device such as a mouse; a monitor; and other peripheral devices. For ease of reference, the method will be described with reference to the hexapod manipulator of FIG. 1.

At a step 302, the surgeon attaches the first ring 12 of the hexapod manipulator 10 to a first segment 100a of a fractured long bone 100 using pins. The surgeon then attaches the second ring 14 to the second segment 100b of the long bone 100. In some methods, the surgeon connects the first bone segment 100a to the first ring 12 in a manner that the bone segment 100a projects substantially normal to a plane formed by the first ring 12. The second ring 14 may be disposed about the second bone segment 100b and attached to the bone segment 100b using pins, in the arrangement shown in FIG. 1. In some examples, the second ring 14 is attached to the bone segment 100b when all six struts are set to have substantially equal lengths, thereby aligning the two rings into predictable, parallel planes.

Once attached, the first ring 12 may be considered and treated as a “Fixed Reference” with grounded mounts, and the second ring 14 may be considered and treated as a “Moving Reference” as described further below. Although the first ring is identified here as the Fixed Reference, it would be apparent to one skilled in the art that either the first or second ring may be either the Fixed Reference or the Moving Reference.

At a step 304, the processing system receives the lengths of the six struts 4 of the hexapod manipulator 1. These may be input by the surgeon or other health care provider. The processing system, such as a computer, is programmed to determine the FKS for the specific manipulator configuration and ultimately output the desired strut lengths that will be used to properly align the bone segments.

At a step 306, the deformity is characterized by the processing system. This may include using digital x-ray images of the bone segments and the hexapod manipulator to determine the location of the bone segments relative to the rings.

For example, digital images of x-rays that depict the deformity and a manipulator relative to the deformity are used to determine the mounting conditions of the manipulator. This technique for gathering data greatly reduces or eliminates the need for a physician to take physical measurements from the x-rays.

Accordingly, to start off, digital x-ray images are generated. These may be at any angle relative to each other, recognizing that images taken at 90 degrees relative to each other will provide greatest accuracy for analysis. From these images, the bone segments may be characterized in computer space with the snapping of lines, clinical evaluation of the rotation, and other bone segment characteristic defining methods. One example of characterizing bone segments is described in U.S. Patent Publication No. 2004/0097922 to Mullaney, incorporated by reference above. Further, using digital x-ray images may be used to characterize the manipulator relative to the bone segments. So long as at least three of the spherically shaped ball joints of the spatial manipulator are shown in the x-ray images, the locations of the relative positions of the manipulator and bone segments may be determined.

In the x-ray images, the spherical or spheroid ball joints appear as ellipsoidal or circular images. These images can be used for distortion correction and scaling if necessary. The images characterize the manipulator in 3D space. If the bone segments are also characterized in the same 3D space, then the bone segments and the hexapod manipulator can be related. The circular images are artifacts within the digital x-ray image. The processing system may use these as anchors to superimpose a digital replica of the manipulator into the digital x-ray images. This digital replica of the spatial manipulator assists in determining the relationship between the actual manipulator and the bone segments.

The processing system disclosed herein may be used to calculate the location of the manipulator to identify and locate the position of the manipulator in digital space using any combination of three balls joints on both rings (i.e., one ball associated with one ring and two balls associated with the other ring) of the six ball joints. This may begin with characterizing the hexapod manipulator as a rigid entity using the ring sizes and known strut lengths. This allows for the determination of the relative positions of whatever balls are found relative to one another which, then allows for the determination of the position and pose of the hexapod manipulator. In some instances, if more than three of the spherical shaped ball joints are visible on the x-ray images, the computer calculates the manipulator location to identify and locate its position multiple times using a different combination of the spherical ball joints each time. The results of the multiple calculations may then be averaged to provide an even more accurate indication of the location and position of the manipulator.

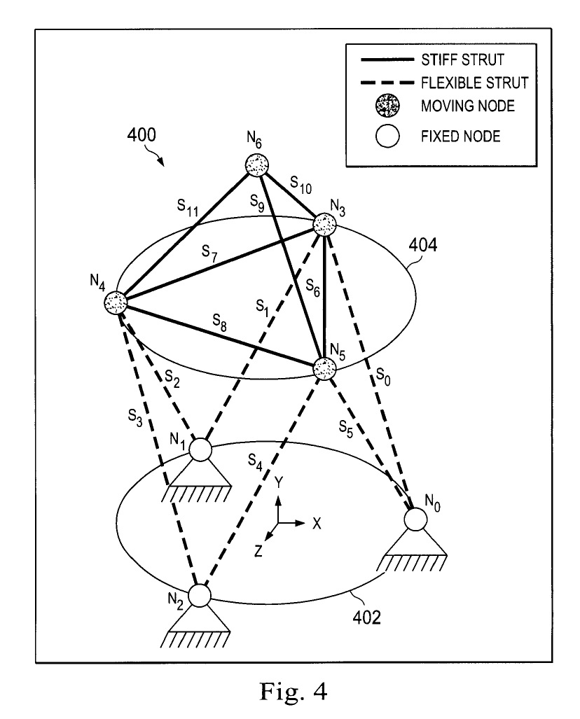

Having characterized the deformity at step 306 in FIG. 3, the next step is determining the proper deformity correction, as indicated at a step 308. At step 308, the processing system determines the proper deformity correction by relating the rings on an individual basis to the fragment to which it each ring is connected to identify an initial position, manipulating the bone fragments to a desired position by moving the Moving Reference ring relative to the Fixed Reference ring, and determining the distance moved from the initial to the desired position. This includes setting up a model, calculating the displacement using a 3 dimensional distance formula, such as the Euclidean distance formula including determining the square-root of the sum of the coordinate differences squared, and moving the distal bone segment and its associated ring in virtual space so that the distal bone segment properly aligns with the proximal bone segment. This is described below with reference to FIGS. 4-7.

FIG. 4 is a schematic representation of a spatial manipulator 400 in computer space without the digitized bone segments in order to explain the principles of the present disclosure. Principally, it comprises a pair a reference rings 402, 404 each containing three ball joints represented by nodes Nx. One of the reference rings 302 is referred to as a Fixed Reference depicted here with the grounded ball joint mounts represented by nodes N0, N1 and N2. The ball joint mounts make up the nodes N0, N1 and N2 and are fixed in a global Cartesian coordinate system x, y, z. These also correspond with the ball joints 18 of the physical hexapod manipulator 10 of FIG. 1. The second reference ring 404 is known as the Moving Reference and also contains three ball joints represented by and making up nodes N3, N4, and N5. These rings 302, 304 typically represent rigid constructs, such as the rings 12, 14 shown in FIG. 1. Strut “elements” in FIG. 4 connect joints or nodes and represent the struts 16 in FIG. 1.

This exemplary model accounts for three ball joints affixed to each ring, with the ball joints being shared by pairs of struts. This model is referred to as a 3×3 hexapod, and is used in this example for simplicity. However, it should be noted that a more preferred model is referred to as a 6×6 hexapod where the ball joints of struts are not shared but are closely paired, with three groups of 2 joints on each ring. This mathematical model can be extended to a 6×6 accounting for the additional stiff elements linking the paired ball joints. Accordingly, in such examples, each ring is modeled as a stiff hexagon rather than a stiff triangle.

For the purpose of this study and for the sake of simplicity a 7^{th} node has been added and referenced by N6. The position of node N6 is fixed relative to the position of nodes N3, N4, and N5 through strut “elements” S9, S10 and S11 in FIG. 4. The positions of the nodes N3, N4, and N5 are also fixed relative to one another in a planar fashion through the use of strut “elements” S6, S7 and S8. Each of the struts “elements” S6, S7, S8, S9, S10 and S11 is considered to a “stiff” element, meaning the position of the nodes connected by the strut “elements” S6, S7, S8, S9, S10 and S11 does not change relative to each other. As can be seen in FIG. 4, each of the strut “elements” S6, S7, S8, S9, S10 and S11 are arranged with a ball joint at each end in order to avoid the need to implement six DOF elements to take into account end moments Mx, My, and Mz. As would be apparent to one skilled in the art, the ball joints render any loading as axial loads. Also, as can be readily noted, the data of interest is the nodal positions and the longitudinal “strains” or actual axial deflections of the elements themselves.

As shown in FIG. 4, strut “elements” S0, S1, S2, S3, S4 and S5 connect nodes N0>N3>N1>N4>N2>N5>N0 and, instead of having fixed lengths, are telescopic in nature. These strut elements, however, are considered flexible and their lengths will be incremented from an initial collection of lengths (or starting lengths) to a desired collection of lengths (or ending lengths).

The ultimate objective will be to determine the coordinate transformation of the Moving Reference ring 404 with respect to the Fixed Reference ring 402. This can be easily achieved once the nodal positions N3, N4 and N5 are known in the coordinate system. A simple cross product between the vectors “struts” connecting any pair of these nodes will yield a normal vector. A subsequent cross product of this normal vector and either of the previous vectors, depending on sign convention, will yield the third mutually normal vector describing the Moving Reference coordinate system.

Constructing the Model

In order to find the nodal positions N3, N4, and N5 that ultimately allow the determination of the coordinate transformation of the Moving Reference ring 404 with respect to the Fixed Reference ring 402, a global stiffness matrix is constructed. This represents a model of the hexapod manipulator. To construct this, a first analysis is performed of each strut individually, and the results of the individual strut analysis are combined to determine the overall global stiffness matrix.

To construct this model, analysis of the position of each strut is individually performed. Here, the struts themselves are considered the “elements” in a finite element solution for finding the relative ring displacement to arrive at the desired bone segment alignment.

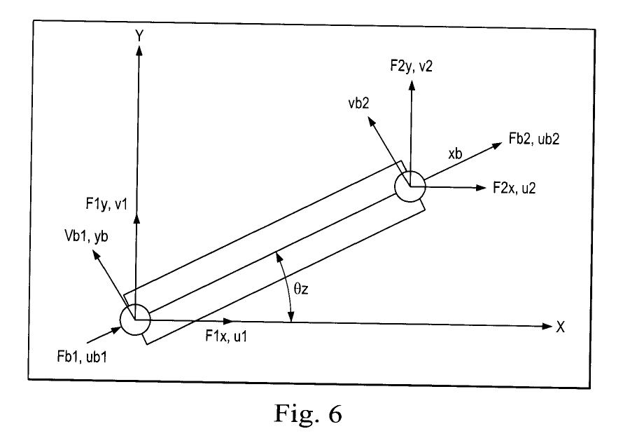

The processing system identifies the location of each element individually, by first considering the relationship between a) the element forces and displacements in element coordinates and b) the Forces and displacements in the global coordinate system. A method of doing this is described with reference to the flow chart in FIG. 5. In accordance with the flow chart, the first step 502 is determining both the forces and displacements of a single element in both the element coordinates and the global coordinate system. This step is discussed with reference to FIG. 6 showing a bar element representing a strut of the hexapod manipulator. In the example shown in FIG. 6, the bar element representing the strut is rotated about an arbitrary reference so that the bar element is oriented at an angle θz about the Z-axis, measured counterclockwise from the positive X-axis, as at step 504. In two dimensions this is simply the following:

$$( \begin{matrix}{ub}_{1} \\ {vb}_{1} \\ {ub}_{2} \\ {vb}_{2}\end{matrix} ) = ( \begin{matrix}cos ( θ e ) & sin ( θ e ) & 0 & 0 \\ - sin ( θ e ) & cos ( θ e ) & 0 & 0 \\ 0 & 0 & cos ( θ e ) & sin ( θ e ) \\ 0 & 0 & - sin ( θ e ) & cos ( θ e )\end{matrix} ) \cdot ( \begin{matrix}{u}_{1} \\ {v}_{1} \\ {u}_{2} \\ {v}_{2}\end{matrix} )$$

In accordance with FIG. 6, this provides both the forces and displacements in both the element and global coordinate system. Of course this is only sufficient for plane trusses, and should be expanded to consider a spatial truss.

In order to consider six displacements and six forces for each element a second rotation must be performed about a global coordinate system axis, as indicated at step 506 in FIG. 5. This example makes a second rotation about the global coordinate system Y-axis.

It should be noted that step 506 does not use the Euler method which would make a second rotation about an axis in the already previously rotated system. Further, because each element is constrained at either end with a ball joint, a third rotation about the longitudinal axis of any of the elements is unnecessary. A rotation about the global Y-axis leaves the previous y locations unaltered. The second rotation taken after the first results in the following sets of equations:

$$( \begin{matrix}cos ( θ {z}_{e} ) & - sin ( θ {z}_{e} ) & 0 \\ sin ( θ {z}_{e} ) & cos ( θ {z}_{e} ) & 0 \\ 0 & 0 & 1\end{matrix} ) \cdot ( \begin{matrix}cos ( θ {y}_{e} ) & 0 & sin ( θ {y}_{e} ) \\ 0 & 1 & 0 \\ - sin ( θ {y}_{e} ) & 0 & cos ( θ {y}_{e} )\end{matrix} ) = ( \begin{matrix}cos ( θ {y}_{e} ) \cdot cos ( θ {z}_{e} ) & - sin ( θ {z}_{e} ) & sin ( θ {y}_{e} ) \cdot cos ( θ {z}_{e} ) \\ cos ( θ {y}_{e} ) \cdot sin ( θ {z}_{e} ) & cos ( θ {z}_{e} ) & sin ( θ {y}_{e} ) \cdot sin ( θ {z}_{e} ) \\ - sin ( θ {y}_{e} ) & 0 & cos ( θ {y}_{e} )\end{matrix} )$$

Step 508 is determining the transformation between the global coordinate system and the element coordinate system. Combining the right side of this equation into a 6×6 matrix to represent both node 1 and node 2 for any given element we get:

$$Te := ( \begin{matrix}\begin{matrix}cos ( θ {y}_{e} ) \cdot \\ cos ( θ {z}_{e} )\end{matrix} & sin ( θ {z}_{e} ) & \begin{matrix}- sin ( θ {y}_{e} ) \cdot \\ cos ( θ {z}_{e} )\end{matrix} & 0 & 0 & 0 \\ \begin{matrix}- cos ( θ {y}_{e} ) \cdot \\ sin ( θ {z}_{e} )\end{matrix} & cos ( θ {z}_{e} ) & \begin{matrix}sin ( θ {y}_{e} ) \cdot \\ sin ( θ {z}_{e} )\end{matrix} & 0 & 0 & 0 \\ sin ( θ {y}_{e} ) & 0 & cos ( θ {y}_{e} ) & 0 & 0 & 0 \\ 0 & 0 & 0 & \begin{matrix}cos ( θ {y}_{e} ) \cdot \\ cos ( θ {z}_{e} )\end{matrix} & sin ( θ {z}_{e} ) & \begin{matrix}- sin ( θ {y}_{e} ) \cdot \\ cos ( θ {z}_{e} )\end{matrix} \\ 0 & 0 & 0 & \begin{matrix}- cos ( θ {y}_{e} ) \cdot \\ sin ( θ {z}_{e} )\end{matrix} & cos ( θ {z}_{e} ) & \begin{matrix}sin ( θ {y}_{e} ) \cdot \\ sin ( θ {z}_{e} )\end{matrix} \\ 0 & 0 & 0 & sin ( θ {y}_{e} ) & 0 & cos ( θ {y}_{e} )\end{matrix} )$$

Where Te is the transformation between the global coordinate system and the element coordinate system for a bar element without rotation about the longitudinal axis of the element itself.

Steps 502-508 are carried out for each strut. Having identified the location of each element individually by considering the relationship between a) the element forces and displacements in element coordinates and b) the Forces and displacements in the global coordinate system, the next step, as shown at step 510 in FIG. 5, is establishing a global stiffness matrix.

At a step 510, the global stiffness matrix is established. This includes computing the stiffness matrix for each element using the following equation:

[Ke]=[Te]^{T}*[Kbe]*[Te]

where Kbe is a 6×6 representation of the bar element stiffness matrix:

$$( \begin{matrix}1 & - 1 \\ - 1 & 1\end{matrix} ) \cdot \frac{Ee \cdot Ae}{he}$$

Ee and Ae are the Modulus and cross sectional area of the element while he is the element length. In 6×6 matrix form Kbe is as follows:

$$Kbe = ( \begin{matrix}1 & 0 & 0 & - 1 & 0 & 0 \\ 0 & 0 & 0 & 0 & 0 & 0 \\ 0 & 0 & 0 & 0 & 0 & 0 \\ - 1 & 0 & 0 & 1 & 0 & 0 \\ 0 & 0 & 0 & 0 & 0 & 0 \\ 0 & 0 & 0 & 0 & 0 & 0\end{matrix} ) \cdot \frac{Ee \cdot Ae}{he}$$

[Ke] can therefore be represented as follows:

$${( \begin{matrix}\begin{matrix}cos ( θ {y}_{e} ) \cdot \\ cos ( θ {z}_{e} )\end{matrix} & sin ( θ {z}_{e} ) & \begin{matrix}- sin ( θ {y}_{e} ) \cdot \\ cos ( θ {z}_{e} )\end{matrix} & 0 & 0 & 0 \\ \begin{matrix}- cos ( θ {y}_{e} ) \cdot \\ sin ( θ {z}_{e} )\end{matrix} & cos ( θ {z}_{e} ) & \begin{matrix}sin ( θ {y}_{e} ) \cdot \\ sin ( θ {y}_{e} )\end{matrix} & 0 & 0 & 0 \\ sin ( θ {y}_{e} ) & 0 & cos ( θ {y}_{e} ) & 0 & 0 & 0 \\ 0 & 0 & 0 & \begin{matrix}cos ( θ {y}_{e} ) \cdot \\ cos ( θ {z}_{e} )\end{matrix} & sin ( θ {z}_{e} ) & \begin{matrix}- sin ( θ {y}_{e} ) \cdot \\ cos ( θ {z}_{e} )\end{matrix} \\ 0 & 0 & 0 & \begin{matrix}- cos ( θ {y}_{e} ) \cdot \\ sin ( θ {z}_{e} )\end{matrix} & cos ( θ {z}_{e} ) & \begin{matrix}sin ( θ {y}_{e} ) \cdot \\ sin ( θ {z}_{e} )\end{matrix} \\ 0 & 0 & 0 & sin ( θ {y}_{e} ) & 0 & cos ( θ {y}_{e} )\end{matrix} )}^{T} \cdot ( \begin{matrix}1 & 0 & 0 & - 1 & 0 & 0 \\ 0 & 0 & 0 & 0 & 0 & 0 \\ 0 & 0 & 0 & 0 & 0 & 0 \\ - 1 & 0 & 0 & 1 & 0 & 0 \\ 0 & 0 & 0 & 0 & 0 & 0 \\ 0 & 0 & 0 & 0 & 0 & 0\end{matrix} ) \cdot ( \begin{matrix}\begin{matrix}cos ( θ {y}_{e} ) \cdot \\ cos ( θ {z}_{e} )\end{matrix} & sin ( θ {z}_{e} ) & \begin{matrix}- sin ( θ {y}_{e} ) \cdot \\ cos ( θ {z}_{e} )\end{matrix} & 0 & 0 & 0 \\ \begin{matrix}- cos ( θ {y}_{e} ) \cdot \\ sin ( θ {z}_{e} )\end{matrix} & cos ( θ {z}_{e} ) & \begin{matrix}sin ( θ {y}_{e} ) \cdot \\ sin ( θ {z}_{e} )\end{matrix} & 0 & 0 & 0 \\ sin ( θ {y}_{e} ) & 0 & cos ( θ {y}_{e} ) & 0 & 0 & 0 \\ 0 & 0 & 0 & \begin{matrix}cos ( θ {y}_{e} ) \cdot \\ cos ( θ {z}_{e} )\end{matrix} & sin ( θ {z}_{e} ) & \begin{matrix}- sin ( θ {y}_{e} ) \cdot \\ cos ( θ {z}_{e} )\end{matrix} \\ 0 & 0 & 0 & \begin{matrix}- cos ( θ {y}_{e} ) \cdot \\ sin ( θ {z}_{e} )\end{matrix} & cos ( θ {z}_{e} ) & \begin{matrix}sin ( θ {y}_{e} ) \cdot \\ sin ( θ {z}_{e} )\end{matrix} \\ 0 & 0 & 0 & sin ( θ {y}_{e} ) & 0 & cos ( θ {y}_{e} )\end{matrix} )$$

Which yields:

$${Kbar}_{e} := ( \begin{matrix}\begin{matrix}{cos ( θ {y}_{e} )}^{2} \cdot \\ {cos ( θ {z}_{e} )}^{2}\end{matrix} & \begin{matrix}cos ( θ {y}_{e} ) \cdot \\ cos ( θ {z}_{e} ) \cdot \\ sin ( θ {z}_{e} )\end{matrix} & \begin{matrix}- cos ( θ {y}_{e} ) \cdot \\ {cos ( θ {z}_{e} )}^{2} \cdot \\ sin ( θ {y}_{e} )\end{matrix} & \begin{matrix}- cos {( θ {y}_{e} )}^{2} \cdot \\ {cos ( θ {z}_{e} )}^{2}\end{matrix} & \begin{matrix}- cos ( θ {y}_{e} ) \cdot \\ cos ( θ {z}_{e} ) \cdot \\ sin ( θ {z}_{e} )\end{matrix} & \begin{matrix}cos ( θ {y}_{e} ) \cdot \\ {cos ( θ {z}_{e} )}^{2} \cdot \\ sin ( θ {y}_{e} )\end{matrix} \\ \begin{matrix}cos ( θ {y}_{e} ) \cdot \\ cos ( θ {z}_{e} ) \cdot \\ sin ( θ {z}_{e} )\end{matrix} & {sin ( θ {z}_{e} )}^{2} & \begin{matrix}- sin ( θ {z}_{e} ) \cdot \\ sin ( θ {y}_{e} ) \cdot \\ cos ( θ {z}_{e} )\end{matrix} & \begin{matrix}- cos ( θ {y}_{e} ) \cdot \\ cos ( θ {z}_{e} ) \cdot \\ sin ( θ {z}_{e} )\end{matrix} & - {sin ( θ {z}_{e} )}^{2} & \begin{matrix}sin ( θ {z}_{e} ) \cdot \\ sin ( θ {y}_{e} ) \cdot \\ cos ( θ {z}_{e} )\end{matrix} \\ \begin{matrix}- cos ( θ {y}_{e} ) \cdot \\ cos {( θ {z}_{e} )}^{2} \cdot \\ sin ( θ {y}_{e} )\end{matrix} & \begin{matrix}- sin ( θ {z}_{e} ) \cdot \\ sin ( θ {y}_{e} ) \cdot \\ cos ( θ {z}_{e} )\end{matrix} & \begin{matrix}{sin ( θ {y}_{e} )}^{2} \cdot \\ {cos ( θ {z}_{e} )}^{2}\end{matrix} & \begin{matrix}cos ( θ {y}_{e} ) \cdot \\ {cos ( θ {z}_{e} )}^{2} \cdot \\ sin ( θ {y}_{e} )\end{matrix} & \begin{matrix}sin ( θ {z}_{e} ) \cdot \\ sin ( θ {y}_{e} ) \cdot \\ cos ( θ {z}_{e} )\end{matrix} & \begin{matrix}- sin {( θ {y}_{e} )}^{2} \cdot \\ {cos ( θ {z}_{e} )}^{2}\end{matrix} \\ \begin{matrix}- {cos ( θ {y}_{e} )}^{2} \cdot \\ {cos ( θ {z}_{e} )}^{2}\end{matrix} & \begin{matrix}- cos ( θ {y}_{e} ) \cdot \\ cos ( θ {z}_{e} ) \cdot \\ sin ( θ {z}_{e} )\end{matrix} & \begin{matrix}cos ( θ {y}_{e} ) \cdot \\ {cos ( θ {z}_{e} )}^{2} \cdot \\ sin ( θ {y}_{e} )\end{matrix} & \begin{matrix}{cos ( θ {y}_{e} )}^{2} \cdot \\ {cos ( θ {z}_{e} )}^{2}\end{matrix} & \begin{matrix}cos ( θ {y}_{e} ) \cdot \\ cos ( θ {z}_{e} ) \cdot \\ sin ( θ {z}_{e} )\end{matrix} & \begin{matrix}- cos ( θ {y}_{e} ) \cdot \\ {cos ( θ {z}_{e} )}^{2} \cdot \\ sin ( θ {y}_{e} )\end{matrix} \\ \begin{matrix}- cos ( θ {y}_{e} ) \cdot \\ cos ( θ {z}_{e} ) \cdot \\ sin ( θ {z}_{e} )\end{matrix} & - {sin ( θ {z}_{e} )}^{2} & \begin{matrix}sin ( θ {z}_{e} ) \cdot \\ sin ( θ {y}_{e} ) \cdot \\ cos ( θ {z}_{e} )\end{matrix} & \begin{matrix}cos ( θ {y}_{e} ) \cdot \\ cos ( θ {z}_{e} ) \cdot \\ sin ( θ {z}_{e} )\end{matrix} & {sin ( θ {z}_{e} )}^{2} & \begin{matrix}- sin ( θ {z}_{e} ) \cdot \\ sin ( θ {y}_{e} ) \cdot \\ cos ( θ {z}_{e} )\end{matrix} \\ \begin{matrix}cos ( θ {y}_{e} ) \cdot \\ {cos ( θ {z}_{e} )}^{2} \cdot \\ sin ( θ {y}_{e} )\end{matrix} & \begin{matrix}sin ( θ {z}_{e} ) \cdot \\ sin ( θ {y}_{e} ) \cdot \\ cos ( θ {z}_{e} )\end{matrix} & \begin{matrix}- {sin ( θ {y}_{e} )}^{2} \cdot \\ {cos ( θ {z}_{e} )}^{2}\end{matrix} & \begin{matrix}- cos ( θ {y}_{e} ) \cdot \\ {cos ( θ {z}_{e} )}^{2} \cdot \\ sin ( θ {y}_{e} )\end{matrix} & \begin{matrix}- sin ( θ {z}_{e} ) \cdot \\ sin ( θ {y}_{e} ) \cdot \\ cos ( θ {z}_{e} )\end{matrix} & \begin{matrix}{sin ( θ {y}_{e} )}^{2} \cdot \\ {cos ( θ {z}_{e} )}^{2}\end{matrix}\end{matrix} ) \cdot \frac{{Ebar}_{e} \cdot {Abar}_{e}}{{Lbar}_{e}}$$

So, therefore relating the Forces generated within a given element follows as:

[Fe]=[Ke][Ue]

Assembling the elements to form the global stiffness matrix results in a [21×21] matrix that when condensed noting the fact that nodes N0, N1 and N3 are fixed in space yields a [12×12] matrix.

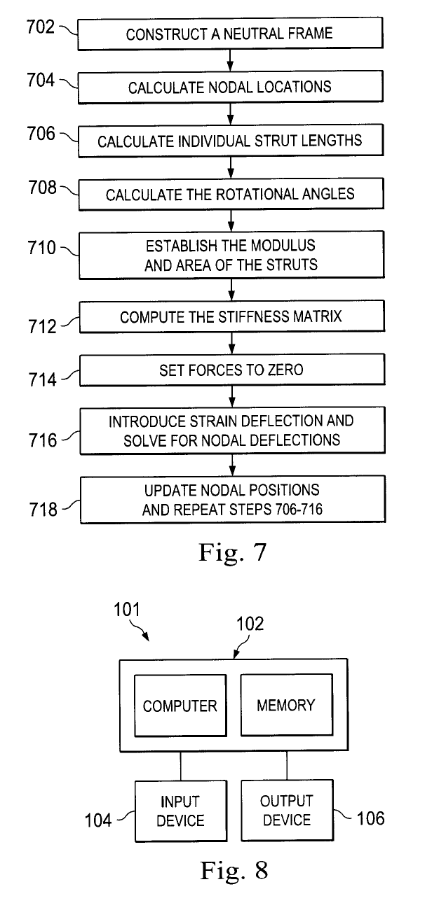

Having set up the global stiffness matrix, the analysis of the complete model may be used to determine the coordinate transformation of the Moving Reference ring 404 with respect to the Fixed Reference ring 402 to ultimately arrive at the desired bone manipulator orientation. The process for doing this is shown in the flow chart in FIG. 7. With reference to FIG. 7, the process may proceed as follows:

At a step 702, the first step is constructing a neutral frame. This may include constructing a frame whose rings are parallel with the Moving Reference ring rotated 60 degrees about the Y-axis with respect to the Fixed Reference ring. In this configuration all of the struts S0 through S5 are equal in length.

At a step 704, the nodal locations are calculated. This is accomplished using the following equation:

$$\begin{matrix}N := [ \begin{matrix}cos ( 0 + Ring Rotati {on}_{0} ) \cdot Ring {Diameter}_{0} \cdot 0.5 \\ Ring Heig {ht}_{0} \\ sin ( 0 + Ring Rotat {ion}_{0} ) \cdot ( {RingDiameter}_{0} \cdot 0.5 ) \\ cos ( \frac{2}{3} \cdot π + Ring Rota {tion}_{0} ) \cdot ( {RingDiameter}_{0} \cdot 0.5 ) \\ Ring Heig {ht}_{0} \\ sin ( \frac{2}{3} \cdot π + Ring Ro ta {tion}_{0} ) \cdot ( {RingDiameter}_{0} \cdot 0.5 ) \\ cos ( \frac{4}{3} \cdot π + Ring Ro ta {tion}_{0} ) \cdot ( {RingDiameter}_{0} \cdot 0.5 ) \\ Ring {Height}_{0} \\ sin ( \frac{4}{3} \cdot π + Ring Rot at {ion}_{0} ) \cdot ( {RingDiameter}_{0} \cdot 0.5 ) \\ cos ( 0 + Ring Rota {tion}_{1} ) \cdot ( {RingDiameter}_{1} \cdot 0.5 ) \\ Ring Heig {ht}_{1} \\ sin ( 0 + Ring Ro ta {tion}_{1} ) \cdot ( {RingDiameter}_{1} \cdot 0.5 ) \\ cos ( \frac{2}{3} \cdot π + Ring Rota {tion}_{1} ) \cdot ( {RingDiameter}_{1} \cdot 0.5 ) \\ RingHeig {ht}_{1} \\ sin ( \frac{2}{3} \cdot π + Ring Rota {tion}_{1} ) \cdot ( {RingDiameter}_{1} \cdot 0.5 ) \\ cos ( \frac{4}{3} \cdot π + Ring Rotati {on}_{1} ) \cdot ( {RingDiameter}_{1} \cdot 0.5 ) \\ Ring Heig {ht}_{1} \\ sin ( \frac{4}{3} \cdot π + Ring Rotati {on}_{1} ) \cdot ( {RingDiameter}_{1} \cdot 0.5 ) \\ 0 \\ Ring Heig {ht}_{2} \\ 0\end{matrix} ] & \\ N = ( \begin{matrix}125 \\ 0 \\ 0 \\ - 62.5 \\ 0 \\ 108.253 \\ - 62.5 \\ 0 \\ - 108.253 \\ 62.5 \\ 125 \\ 108.253 \\ - 125 \\ 125 \\ 1.531 \times {10}^{- 14} \\ 62.5 \\ 125 \\ - 108.253 \\ 0 \\ 200 \\ 0\end{matrix} ) & \end{matrix}$$

At a step 706, the individual strut lengths are calculated:

$$\begin{matrix}L := [ \begin{matrix}\sqrt{{( {N}_{9} - {N}_{0} )}^{2} + {( {N}_{10} - {N}_{1} )}^{2} + {( {N}_{11} - {N}_{2} )}^{2}} \\ \sqrt{{( {N}_{9} - {N}_{3} )}^{2} + {( {N}_{10} - {N}_{4} )}^{2} + {( {N}_{11} - {N}_{5} )}^{2}} \\ \sqrt{{( {N}_{12} - {N}_{3} )}^{2} + {( {N}_{13} - {N}_{4} )}^{2} + {( {N}_{14} - {N}_{5} )}^{2}} \\ \sqrt{{( {N}_{12} - {N}_{6} )}^{2} + {( {N}_{13} - {N}_{7} )}^{2} + {( {N}_{14} - {N}_{8} )}^{2}} \\ \sqrt{{( {N}_{15} - {N}_{6} )}^{2} + {( {N}_{16} - {N}_{7} )}^{2} + {( {N}_{17} - {N}_{8} )}^{2}} \\ \sqrt{{( {N}_{15} - {N}_{0} )}^{2} + {( {N}_{16} - {N}_{1} )}^{2} + {( {N}_{17} - {N}_{2} )}^{2}} \\ \sqrt{{( {N}_{9} - {N}_{15} )}^{2} + {( {N}_{10} - {N}_{16} )}^{2} + {( {N}_{11} - {N}_{17} )}^{2}} \\ \sqrt{{( {N}_{9} - {N}_{12} )}^{2} + {( {N}_{10} - {N}_{13} )}^{2} + {( {N}_{11} - {N}_{14} )}^{2}} \\ \sqrt{{( {N}_{15} - {N}_{12} )}^{2} + {( {N}_{16} - {N}_{13} )}^{2} + {( {N}_{17} - {N}_{14} )}^{2}} \\ \sqrt{{( {N}_{9} - {N}_{18} )}^{2} + {( {N}_{10} - {N}_{19} )}^{2} + {( {N}_{11} - {N}_{20} )}^{2}} \\ \sqrt{{( {N}_{12} - {N}_{18} )}^{2} + {( {N}_{13} - {N}_{19} )}^{2} + {( {N}_{14} - {N}_{20} )}^{2}} \\ \sqrt{{( {N}_{15} - {N}_{18} )}^{2} + {( {N}_{16} - {N}_{19} )}^{2} + {( {N}_{17} - {N}_{20} )}^{2}}\end{matrix} ] & \\ L = ( \begin{matrix}176.777 \\ 176.777 \\ 176.777 \\ 176.777 \\ 176.777 \\ 176.777 \\ 216.506 \\ 216.506 \\ 216.506 \\ 145.774 \\ 145.774 \\ 145.774\end{matrix} ) • & \end{matrix}$$

At a step 708, the system calculates the rotation angles θz, θy required by the transformation matrix [Te] with the initial nodal locations and strut lengths:

$$θ z := ( \begin{matrix}a sin ( \frac{{N}_{10}}{{L}_{0}} ) \\ a sin ( \frac{{N}_{10}}{{L}_{1}} ) \\ a sin ( \frac{{N}_{13}}{{L}_{2}} ) \\ a sin ( \frac{{N}_{13}}{{L}_{3}} ) \\ a sin ( \frac{{N}_{16}}{{L}_{4}} ) \\ a sin ( \frac{{N}_{16}}{{L}_{5}} ) \\ \frac{a sin ( {N}_{10} - {N}_{16} )}{{L}_{6}} \\ \frac{a sin ( {N}_{13} - {N}_{10} )}{{L}_{7}} \\ \frac{a sin ( {N}_{16} - {N}_{13} )}{{L}_{8}} \\ a sin ( \frac{{N}_{19} - {N}_{10}}{{L}_{9}} ) \\ a sin ( \frac{{N}_{19} - {N}_{13}}{{L}_{10}} ) \\ a sin ( \frac{{N}_{19} - {N}_{16}}{{L}_{11}} )\end{matrix} ) \qquad θ z \cdot \frac{180}{π} = ( \begin{matrix}45 \\ 45 \\ 45 \\ 45 \\ 45 \\ 45 \\ 0 \\ 0 \\ 0 \\ 30.964 \\ 30.964 \\ 30.964\end{matrix} ) •$$

At a step 710, the modulus and cross-sectional area Ee Ae products are established for the given Struts to differentiate stiff from flexible members.

At a step 712, the Global stiffness matrix [K] is computed for the initial condition.

At a step 714, the forces [Fi] are set equal to [0]. A small strain is introduced into the struts (he+Δs) at as step 716. The condensed system is then solved for the nodal deflections.

At a step 718, the nodal positions are updated and steps 606-616 are repeated. This continues until the deflection is sufficiently small, i.e. gradient approaches zero, indicating that the deflected position has arrived at the desired position.

The above algorithm would be expected to provide a smooth mapping of the nodal positions as the struts are adjusted from the initial position to the final settings without any load applied. If it is desired a fixed or changing displacement could be applied to node N6 and the resultant forces could be obtained using the described methods using the principle of minimum total potential energy. This would not only result in a positional analysis but also a stiffness result as well.

Returning to the method of FIG. 3, at a step 310, the processing system outputs desired strut lengths that would align the first and second bone segments in the manner desired. In some embodiments, the processing determines the difference between the input starting strut lengths and the calculated ending strut lengths and outputs the desired strut lengths in incremental steps that the surgeon can follow to align the first and second bone segments.

At a step 312, the surgeon manipulates the struts to have the desired lengths, thereby aligning the first and second bone segments for healing.

In this example the relative initial or beginning position of the rings is known because the struts all have the same length. In other embodiments, however, the rings are attached to the respective bone segments in a non-orthogonal condition. In these instances, the processing system determines the initial relative position of the rings based on the initial strut lengths, which may be input into the processing system.

In some embodiments, the manipulation is accomplished gradually, with the processing system outputting the data representing the desired length for each strut in small increments, with each increment bringing the rings, and likewise the bone segments, closer to the desired position. This may aid the surgeon by providing objective step-by-step data that guides the rings to the desired position, instead of merely providing the desired ending strut lengths. In one such system, the processing system uses the output data of one iteration as the initial or starting data of the following iteration.

Once the output data is determined by the processing system, the surgeon manipulates the telescoping struts to change the strut lengths per the output data and move the second ring relative to the first ring toward or to the desired position. In most cases, the manipulation takes place over time while the bones are healing. In one example, the device is manipulated to lengthen a limb, where a deficit of bone exists and the bones are initially set short. As the bone segments start to form callus, the manipulator is controlled to “stretch” while the newly formed bone tissue is still pliable, thereby elongating the bone during the healing process.

Because the computer program product is operable on a standard processing system that may be located in a doctor's office or in an operating room, the surgeon has immediate, secure, locally-based accessibility to the desired data. Furthermore, because the computer program product can be executed on a standard processing system, expensive equipment can be minimized and the application may be installed and operated on systems that may already be currently located in operating rooms, recovery centers, outpatient facilities and other healthcare facilities. Still further, as an alternative to or in addition to a surgeon, other health care providers can calculate desired adjustments for the manipulator. This can be at the time of initial implantation or as part of an adjustment during the healing process.

In some embodiments, a transducer may be associated with each of the struts to monitor and provide loading feedback that may be used to evaluate healing, status, or other information about the hexapod or patient. In some embodiments, these transducers may include springs that measure deflection, strain gauges, piezoelectric elements, inchworm motors, actuators, or other systems that provide or detect loading on the hexapod and bone segments. Accordingly, in some embodiments, the struts are dynamic and the system processes deflection to calculate a loading readout.

FIG. 8 is a block diagram of an exemplary processing system 101 that may store and/or execute computer program product carrying out at least a portion of the methods described herein. As indicated above, the processing system 101 may be a fixture in a doctor's office, an operating room, or may be disposed elsewhere. In accordance with this, the processing system 101 may be a commercially obtainable personal computer. Referring to FIG. 8, the exemplary processing system 101 has a case 102 including a processor and memory. The processor may be any suitable processor or microprocessor. The memory collectively represents several different types of memory that are present in the processing system 101. For example, the memory includes a hard disk drive, a “flash” random access memory (RAM), a volatile random access memory, a read only memory (ROM), and so forth. The memory stores a variety of programs that can be executed by the processor for carrying out the methods described herein, including calculating the positions of the hexapod manipulator using non-linear methods, stiffnesses, and deflections as described herein.

The processing system 101 includes an input device 104 such as a keyboard, mouse, or touch screen, among other known input devices. In some embodiments, the processing system is associated with an x-ray imaging system configured to snap x-ray images in a digital format for analysis by the processing system. The processing system 101 also includes an output device 106, such as a display, a printer, an audible signal generator, or other device, for example. The input and output devices provide an interface for use by the health care provider.

The memory and processor of the processing system 102 may be configured in a manner to receive data from the input device 106 and process the data to determine a desired condition or relative positions of the rings of the hexapod, as determined by strut length. The strut length corresponding to the desired position is then output from the processing system at the output device 106. For example, the strut lengths may be displayed on the output device, called out by an audible output device, or otherwise outputted to relay the data to the surgeon.

In some embodiments, the memory and processor are conventional components, but include computer executable programs that evaluate the input data and output calculated data to the display that may be interpreted by an operator, such as a physician or technician. For example, the memory and processor may include a computer executable program that performs the assessment described above to determine the strut lengths that will move the first and second rings to relative desired positions.

The foregoing has outlined features of several embodiments. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions and alterations herein without departing from the spirit and scope of the present disclosure.

I claim:

1. A method of treating a fractured bone using a hexapod manipulator, the hexapod manipulator having a first ring and a second ring, the first and second rings being connected by six telescopic struts, the method comprising:
determining a current position of the second ring relative to the first ring;
determining a desired position of the second ring relative to the first ring;
computing with a processing system the difference between the current position and the desired position of the second ring relative to the first ring using a linear approach to a nonlinear problem.

2. The method of claim 1, comprising:
constructing with the processing system a neutral frame having a Moving Reference ring and a Fixed Reference ring with nodal locations representing ball joints on the hexapod manipulator; and
calculating the nodal locations of the Moving Reference ring using a global stiffness matrix K.

3. The method of claim 1, comprising outputting at least one of:
the difference in strut lengths between the current position and the desired position, and
a strut set value representative of the desired position.

4. The method of claim 1, including determining the forces and displacements of a single element in both an element coordinate system and in a global coordinate system, the element coordinate system relating to a single strut position and the global coordinate system being the coordinate system used to reference the manipulator.

5. The method of claim 1, wherein computing with a processing system the difference between the current position and the desired position comprises:
setting applied forces equal to zero and introducing a small strain into the struts; and
solving for nodal deflections.

6. The method of claim 5, including updating the nodal positions to reflect new positions as a result of the deflections and again solving for nodal deflections at the new positions.

7. A method of determining the difference between a first initial position of a hexapod fixator and a second desired position of the fixator, the fixator having six struts extending between and connecting two fixator rings, the method comprising:
generating a first digital x-ray image of a plurality of bone segments and the hexapod fixator;
generating a second digital x-ray image of a plurality of bone segments and the hexapod fixator, the second digital x-ray image being taken from a angle relative to the first digital x-ray image;
receive data indicative of the lengths of the six struts of the hexapod manipulator;
determining a first position of nodes representative of the connection location of the rings and struts of the hexapod manipulator relative to a coordinate system in space;
deflecting the struts a first amount in the direction of the desired position of the manipulator;
determining a second position of nodes representative of the connection location of the rings and deflected struts of the hexapod manipulator relative to the coordinate system in space; and
deflecting the struts a second amount in the direction of the desired position of the manipulator.

8. The method of claim 7, including repeating the determining and deflecting steps until the determined position of nodes representative of the connection location of the rings and struts corresponds to the second desired position of the fixator, the determined position of the nodes being the final position of the nodes.

9. The method of claim 8, comprising calculating the difference between the first position of the nodes and the final position of the nodes and outputting the difference to a health care provider.

10. The method of claim 7, wherein deflecting the struts is accomplished independent of loading applied to the struts.

11. The method of claim 7, wherein the step of determining the first position of nodes includes:
determining a first rotation of a bar element representative of a single strut in both an element coordinate system relating to only the bar element and a global two-dimensional coordinate system relating to the hexapod manipulator;
determining a second rotation of the bar element about a single axis in the global coordinate system, the single axis being different than the two dimensions used to determine the first rotation of the bar element.

12. The method of claim 11, including the step of computing a global stiffness matrix for the manipulator.

13. The method of claim 12, including setting applied forces to zero to introduce small strain for the condensed system.

14. The method of claim 11, including the step of determining the first and second rotations for each of the six struts.

15. The method of claim 7, including the steps of:
introducing strain deflections; and
calculating a global stiffness matrix to determine the level of nodal deflections.

16. An apparatus comprising a tangible computer-readable storage medium storing a computer program for execution by at least one processor, wherein the program determines a position of a hexapod manipulator having six struts, the program when executed:
receives input data representative of initial strut lengths;
determines an initial orientation a first ring relative to a second ring based in part of the received input data;
establishes a Modulus and cross sectional area for the struts;
computes a stiffness matrix based in part on the Modulus and cross sectional area of the struts;
calculates desired strut lengths that displace the second ring to a desired position; and
outputs the calculated desired strut lengths.

17. The apparatus of claim 16, comprising:
determining a coordinate transformation of a Moving Reference ring with respect to a Fixed Reference ring of the hexapod manipulator.

18. The apparatus of claim 17, comprising:
determining nodal positions of the moving reference ring in a global coordinate system.

19. The apparatus of claim 18, including establishing a global stiffness matrix based on a transformation between the global coordinate system and a element coordinate system, the element being representative of a strut of the hexapod manipulator.

20. A method of treating a fractured bone using a hexapod manipulator, the hexapod manipulator having a first ring and a second ring, the first and second rings being connected by six telescopic struts, the method comprising:
placing the first ring about a first bone segment and fixing the position of the first bone segment relative to the first ring and placing the second ring about the second bone segment and fixing the position of the second bone segment relative to the second ring, the first and second rings being connected at ball joints by the struts;
determining a current position of the second ring relative to the first ring;
determining a desired position of the second ring relative to the first ring;
inputting data indicative of the strut lengths into a processing system;
computing desired strut lengths with the processing system, the desired strut lengths corresponding with the desired position of the second ring relative to the first ring, the step of computing desired strut lengths comprising:
constructing with the processing system a neutral frame having a Moving Reference ring and a Fixed Reference ring with nodal locations representing the ball joints of the placed hexapod manipulator;
calculating the nodal locations of the Moving Reference ring;
calculating the individual strut lengths;
calculating a rotation angle of the Moving Ring relative to the Fixed Reference ring;
establishing Ee Ae products to differentiate stiff and flexible members;
computing the global stiffness matrix K for the initial condition;
setting the forces equal to zero and introduce a small strain into the struts and solving for nodal deflections;
update the nodal positions to reflect new positions as a result of the deflections; and
adjusting the struts so the actual strut lengths correspond to the desired strut lengths to position the first and second bone segments for healing.
